(19) United States
(12) Patent Application Publication
(10) Pub. No.: US 2017/0100057 A1
(43) Pub. Date: Apr. 13, 2017

(54) PORTABLE NDIR BREATH ACETONE MEASUREMENT APPARATUS WITH SUB-PPM ACCURACY

(71) Applicant: ORIENTAL SYSTEM TECHNOLOGY INC., HSINCHU (TW)

(72) Inventor: Chien-Hsun WANG, Hsinchu City (TW)

(21) Appl. No.: 14/880,607

(22) Filed: Oct. 12, 2015

(51) Int. Cl. A61B 5/08; G01N 21/3504; A61M 39/24; G01N 33/497; A61B 5/097; A61B 5/00

(52) U.S. Cl. CPC A61B 5/082; A61B 5/097; A61B 5/0082; A61B 5/7278; A61M 39/24; A61B 5/7225; A61B 5/742; G01N 33/497; G01N 21/3504; A61B 2560/0431; A61B 2560/0223; G01N 2033/4975

(57) ABSTRACT

A portable NDIR breath acetone measurement apparatus includes a portable housing formed with an optical cavity, which is housed in a receiving chamber for containing a gas sample taken from an exhaled breath of a human subject. A control unit controls the temperature in the receiving chamber to a desired temperature, and controls an IR emitter disposed in the receiving chamber to stably emit IR spectrum radiation propagating along a zigzag path toward an IR detector, which is operated at the desired temperature to generate, in response to detection of the IR spectrum radiation, an IR signal that is processed and analyzed by a signal processing unit to obtain a measurement result indicative of the concentration of acetone gas contained in the gas sample.

### FIELD

The disclosure relates to breath acetone measurement and more particularly to a breath acetone measurement apparatus using non-dispersive infrared (NDIR) techniques.

### BACKGROUND

Diabetes has become a common disease. According to International Diabetes Federation (IDF) report in 2013, there were more than 382 million people with diabetes. The World Health Organization (WHO) estimates that the number of people with diabetes will grow to 438 million by the year 2030. In the United States, 29.1 million people have diabetes in the age group of 20-79 in the year 2012. Approximately 1.25 million American children and adults have type 1 diabetes (also called insulin dependent diabetes mellitus (IDDM)), which is a disease of children and adults for which there currently is no adequate means for treatment or prevention.

At least four hundred kinds of volatile organic compounds (VOCs) are found in the human breath. The concentrations of such VOCs are usually measured to be at sub-ppm levels or even lower for healthy human beings. Abnormal concentrations of the breath VOCs are reported to correlate with unhealthy conditions, for instance, acetone gas for diabetes and ammonia gas for renal-disease. Hence, the VOCs in the human breath can potentially be applied as disease-specific biomarkers for non-invasive early detection or monitoring of a variety of diseases.

Higher acetone concentrations ranging from about 1.25 ppmv to about 2.4 ppmv could be detected in an exhaled breath from those who are diabetic, while, theoretically, the acetone concentration for an exhaled breath from a healthy person is typically less than about 0.83 ppmv. However, since acetone could be produced via the fatty acid oxidation in human bodies, excessive acetone circulating in the blood systems may be excreted from the lungs. As a result, in reality, the acetone concentrations ranging from about 0.2 ppmv to about 1.8 ppmv say be detected in an exhaled breath from a healthy person.

For non-invasive monitoring or diagnosis of type-1 diabetic patients, development of a novel breath acetone measurement apparatus, which is compact, portable, inexpensive, and capable of achieving acetone measurement with sub-ppm accuracy, is much desirable.

### SUMMARY

Therefore, an object of the disclosure is be provide a portable breath acetone measurement apparatus using non-dispersive infrared (NDIR) techniques that can achieve acetone measurement with sub-ppm accuracy.

According to the disclosure, a portable non-dispersive infrared (NDIR) breath acetone measurement apparatus includes a portable housing, an IR emitter, a control unit, an IR detector and a signal processing unit.

The portable housing is configured with a receiving chamber that is formed with an optical cavity for containing a gas sample taken from an exhaled breath of a human subject.

The IR emitter is disposed in the receiving chamber for emitting IR spectrum radiation at an IR absorption band of acetone into the optical cavity.

The control unit is connected electrically to the IR emitter. The control unit is configured to control the temperature in the receiving chamber to a desired specific temperature that is higher than ambient temperature of the housing and to control the IR emitter to stabilize the IR spectrum, radiation emitted thereby.

The IR detector is disposed in the optical cavity, and is arranged so that the IR spectrum radiation emitted from the IR emitter propagates to the IR detector along a zigzag optical path that is defined between the IR emitter and the IR detector. The IR detector is operated at the desired specific temperature to detect the IR radiation in the optical cavity so as to generate an IR signal that has a wavelength within a wavelength range around a specific IR absorption wavelength of acetone.

The signal processing unit is connected electrically to the IR detector for receiving the IR signal there from, The signal processing unit is configured to process and analyze the IR signal so as to obtain a measurement result indicative of the concentration of acetone gas contained in the gas sample.

### BRIEF DESCRIPTION OF THE DRAWINGS

Other features and advantages of the disclosure will become apparent in the following detailed description of the embodiments with reference to the accompanying drawings, of which:

FIG. 1 is a schematic block diagram illustrating operative relationships among components of the embodiment of a portable NDIR breath acetone measurement apparatus according to the disclosure;

FIG. 2 is a schematic diagram illustrating configuration of a portable housing, a mouth piece, a water vapor absorption filter, a receiving chamber, an IR emitter, an IR detector and a pressure sensor of the embodiment;

FIG. 3A is a schematic partially sectional view showing the IR emitter of the embodiment;

FIG. 3B is a schematic too view showing the IR emitter of the embodiment without a metal cap and an IR filter;

FIG. 4 is a schematic circuit block diagram illustrating the relationship among the IR emitter, an IR emitter driver circuit and a microprocessor of the embodiment;

FIG. 5 is a plot exemplarily illustrating the IR absorption spectrum for acetone;

FIG. 6 is a plot exemplarily illustrating the relationships between resistance and working temperature of the IR emitter in original and aged states;

FIG. 7 illustrates an example of a polynomial fit curve obtained by the embodiment when operating in a calibration mode; and

FIG. 8 is a plot exemplarily illustrating the relationship between the ambient temperature and variation of the IR spectrum radiation emitted by the IR emitter.

### DETAILED DESCRIPTION

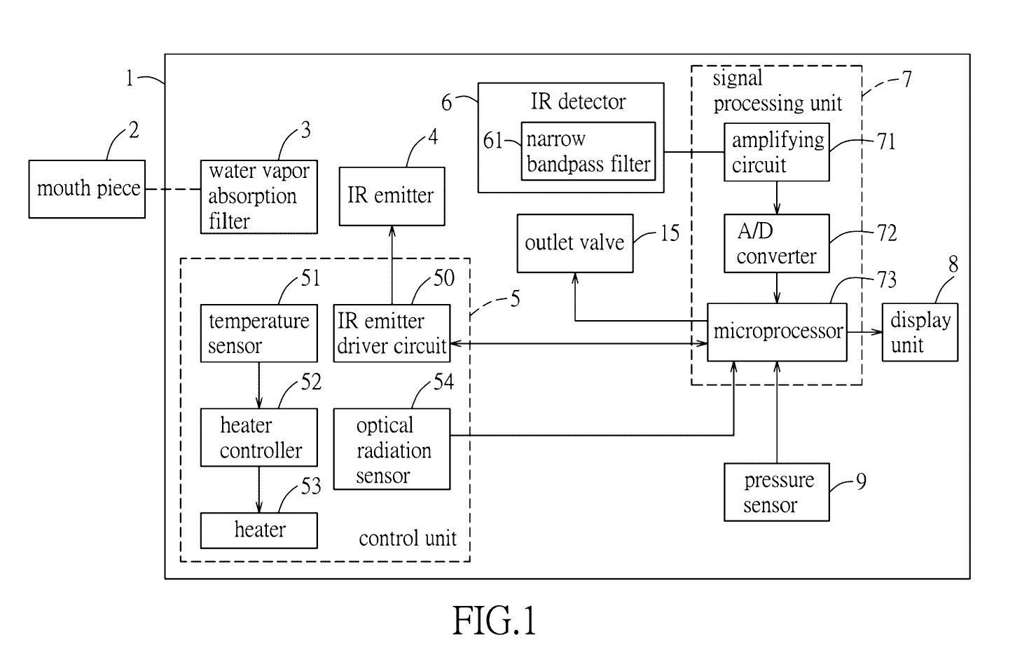

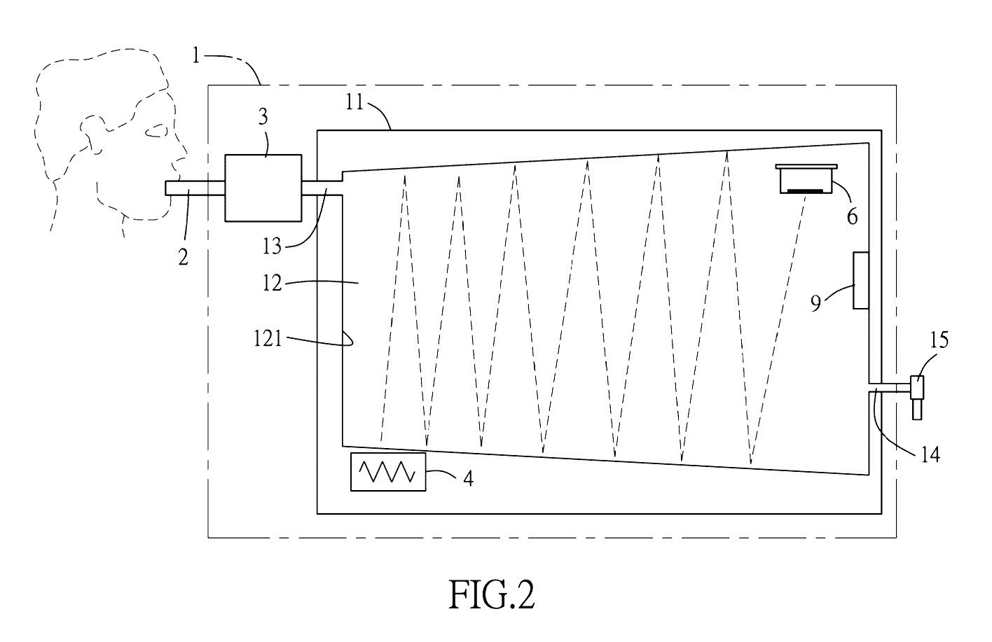

Referring to FIGS. 1 and 2, the embodiment of a portable non-dispersive infrared (NDIR) breath acetone measurement apparatus according to this disclosure is shown to include a portable housing 1, a mouth piece 2, a water vapor absorption filter 3, an IR emitter 4, a control unit 5, an IR detector 6, a signal processing unit 7, a display unit 8 and a pressure sensor 9.

As shown in FIG. 2, the portable housing 1 is configured with a receiving chamber 11 that is formed with an optical cavity 12 for containing a gas sample taken from an exhaled breath of a human subject. In this embodiment, the optical cavity 12 is defined by a highly IR-reflective inner wall surface 121 of the housing 1. The highly IR-reflective inner wall surface 121 is coated with, for example, gold (Au). The receiving chamber 11 has an inlet 13 in fluid communication with the optical cavity 12 for the inflow of gas into the optical cavity 12, and an outlet 14 in fluid communication with the optical cavity 12 for the outflow of gas from the optical-cavity 12. It is noted that the outlet 14 is provided with an outlet valve 15.

The mouth piece 2 is mounted to the housing 1 for collecting the exhaled breath of the human subject.

The water vapor absorption filter 3 is disposed in the housing 1, and is connected fluidly to the mouth piece 2 and to the optical cavity 12 through the inlet 13 for filtering water vapor out of the exhaled breath collected through the mouth piece 2.

It is noted that, during sampling of the gas sample from the exhaled breath filtered by the water vapor absorption filter 3, the outlet valve 15 is controlled in a manner that an end tidal portion of the filtered exhaled breath is kept in the optical cavity 12 to serve as the gas sample.

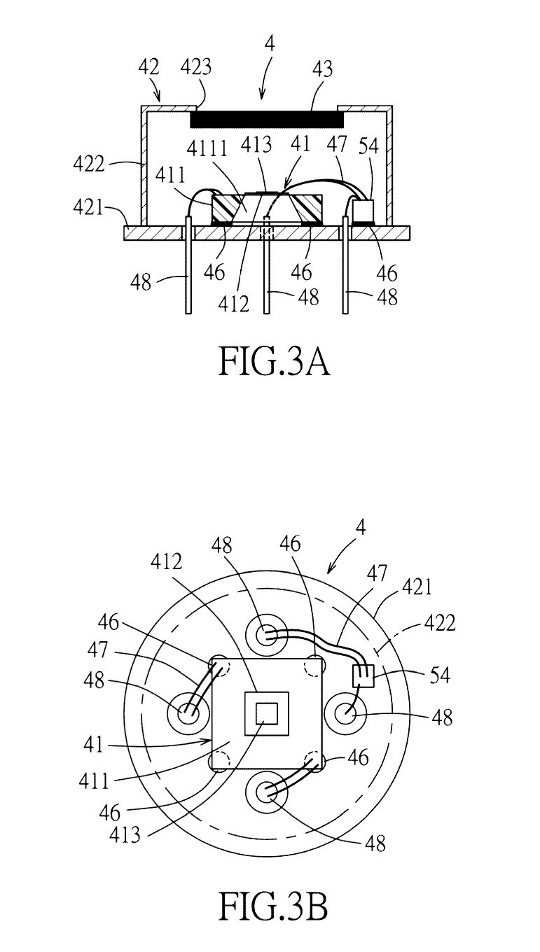

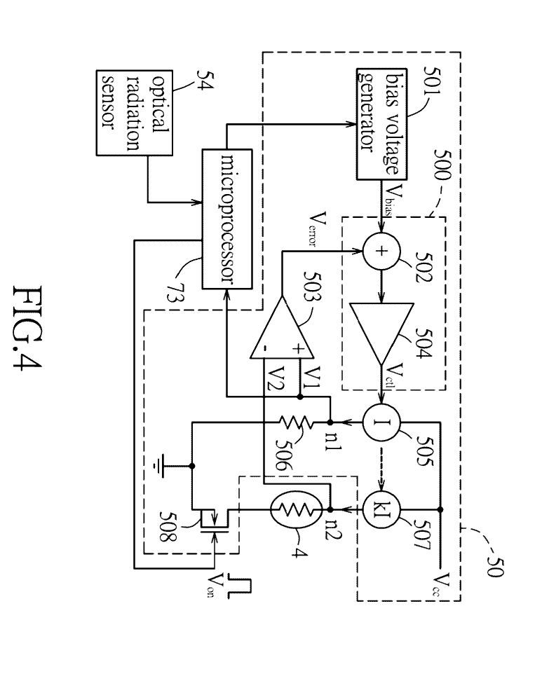

The IR emitter 4 is disposed in the receiving chamber 11 (see FIG. 2) for emitting IR spectrum radiation at an IR absorption band of acetone into the optical cavity 12. Due to the highly IR-reflective inner wall surface 121 defining the optical cavity 12, the IR spectrum radiation emitted by the IR emitter 4 propagates along a zigzag optical path indicated by a dash-line of FIG. 2. It is noted that the length of the zigzag optical path is greater than, for example, 200 cm. In this embodiment, the IR emitter 4 is, for example, a microelectromechanical systems (MEMS)-based IR emitter. Referring to FIGS. 3A and 3B, the IR emitter 4 includes an emitter chip 41, an IE filter 43, and an enclosure 42 for housing the emitter chip 41 and the IR filter 43 therein.

In this embodiment, the enclosure 42 has a metal base 421, and a metal cap 422 connected with the metal base 421 through welding or soldering techniques. The metal base 421 is a so-called header, and is provided insulatingly with a plurality of conductive leads 48 extending therethrough. The metal cap 422 is formed with a window opening 423.

The emitter chip 41 is used to emit IR radiation. In this embodiment, the emitter chip 41 includes a silicon substrate 411, a membrane 412 and a MEMS heating element 413. The silicon substrate 411 has a top surface, a bottom surface attached to the metal base 421 with adhesive glue 46, and a central cavity 4111 extending from the top surface to the bottom surface. The membrane 412 is suspended in the central cavity 4111. The MEMS heating element 413 is disposed on the membrane 412 for heating the membrane 412 up to a high temperature (e.g. 700° C.) so as to generate the IR radiation. The emitter chip 41 is connected electrically to corresponding conductive leads 48 through bonding wires 47.

The IR filter 43 is attached internally to the metal cap 422 to cover sealingly the window opening 423, and is configured to filter the IR radiation emitted by the emitter chip 41 so as to allow the IR spectrum radiation at the absorption band of acetone to radiate out of the enclosure 42 through the window opening 423.

It is noted that, in this embodiment, an optical radiation sensor 54 is disposed in the enclosure 4, and is attached to the metal base 421 with the adhesive glue 46 for sensing the IR radiation emitted by the emitter chip 41 to generate a sensing output. The optical radiation sensor 54 is connected electrically to corresponding conductive leads 48 through bonding wires 47, as best shown in FIG. 3B.

In this embodiment, the IR emitter 4 and the optical radiation sensor 54 may be connected electrically to a required wiring (not shown) through the conductive leads 48.

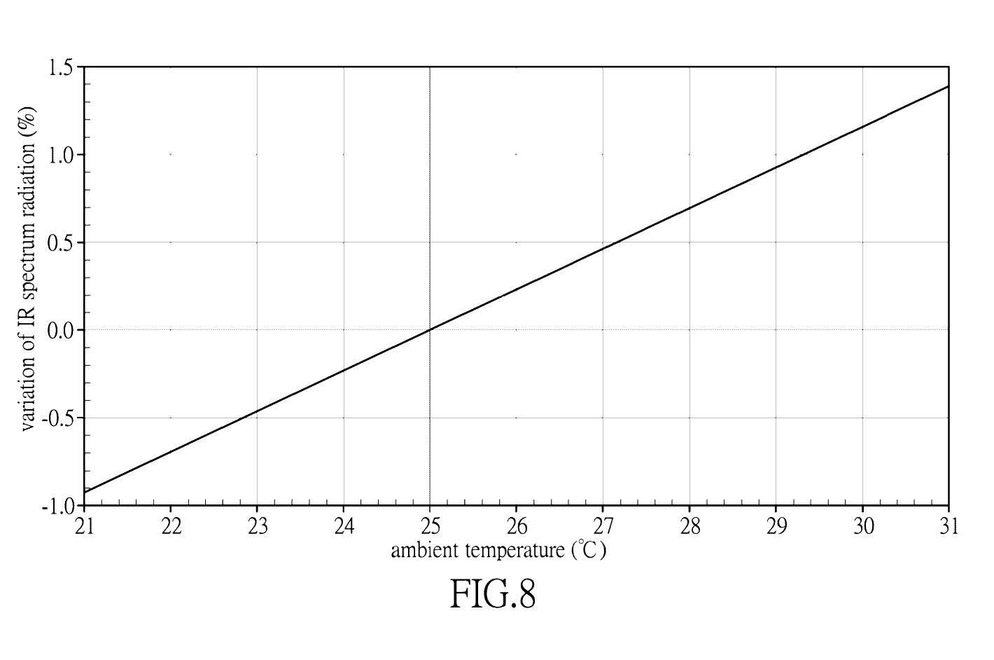

Typically, a conventional driver (not shown) for the IR emitter 4 is based on a fixed power that is susceptible to ambient temperature effect. As an example of the IR emitter 4 operating at a working temperature of 1000° C., when the ambient temperature changes from 25° C. to 21° C., the IR spectrum radiation emitted by the IR emitter 4 may reduce by about one percent (1%), as shown in FIG. 8, which may cause an acetone measurement change of about 1 ppm. Therefore, in order to stabilize the IR spectrum radiation emitted by the IR emitter 4, it is important for the IR emitter 4 to be unaffected by the ambient temperature effect.

Referring back to FIGS. 1 and 2, in order to overcome the above ambient temperature effect for the IR emitter 4, the control unit 5 is configured to control the temperature in the receiving chamber 11 to be a desired specific temperature, for example, 45° C., that is higher than an ambient temperature of the housing 1. Further, the control unit 5 further controls the IR emitter 4 to stabilize the IR spectrum radiation emitted thereby using innovation control for the resistance of the IR emitter 4.

In this embodiment, the control unit 5 may be disposed in the receiving chamber 11 (not illustrated in FIG. 2), and includes a temperature sensor 51, a heater 53, a heater controller 52 and an IR emitter driver circuit 50 (FIG. 1).

The temperature sensor 51 is used to sense the temperature in the receiving chamber 11 to generate an output indicative of the temperature in the receiving chamber 11.

The heater 53 is used to heat the receiving chamber 11.

The heater controller 52 is connected electrically to the temperature sensor 51 and the heater 53. The heater controller 52 receives the output from the temperature sensor 51, and controls the heater 53 based on the output to maintain the temperature in the receiving chamber 11 at the desired specific temperature.

The IR emitter driver circuit 50 is coupled to the IR emitter 4, for example, through the external wiring and the corresponding conductive leads 48 (FIG. 3B) for providing a driving current thereto. Referring to FIG. 4, in this embodiment, the IR emitter driver circuit 50 includes a bias voltage generator 501, a summing amplifier 500, a voltage comparator 503, a voltage controlled current source 505, a mirrored current source 507, a reference sensing resistor 506 and a switch 508.

The voltage controlled current source 505 receives a supply voltage (V_{cc}) and is configured to generate a reference current (I) based on a control voltage (V_{ct1}),

The mirrored current source 507 receives the supply voltage (V_{cc}) and is coupled to the IR emitter 4 for supplying thereto the driving current that is k times the reference current (I), i.e., kI, where k is an amplification factor.

The reference sensing resistor 506 is coupled between the voltage controlled current source 505 and ground to allow the reference current (I) from the voltage controlled current source 505 to flow therethrough.

The switch 508 is coupled between the IR emitter 4 and ground. In use, the switch 508 is controlled to be conducting so as to allow supply of the driving current (kI) from the mirrored current source 507 to the IR emitter 4. In this embodiment, the switch 508 is, for example, an NMOS transistor that has a control end for receiving a control voltage (Vₒₙ) such that the NMOS transistor is operable to be conducting or non-conducting in response to the control voltage (Vₒₙ).

The voltage comparator 503 has a non-inverting input end coupled to a first common node (n1) between the voltage controlled current source 505 and the reference sensing resistor 506 for receiving a voltage (V1) across the reference sensing resistor 506, an inverting input end coupled to a second common node (n2) between the mirrored current source 507 and the IR emitter 4 for receiving a voltage (V2) across the IR emitter 4, and an output end. The voltage comparator 503 compares the voltages (V1, V2) to generate a comparison output (Vₑᵣᵣₒᵣ), and outputs the comparison output (Vₑᵣᵣₒᵣ) at the output end.

The bias voltage generator 501 is configured to generate a bias voltage (V_{bias}) that is controlled by microprocessor 73.

The summing amplifier 500 is coupled to the voltage controlled current source 505, the output end of the voltage comparator 503 and the bias voltage generator 501. The summing amplifier 500 is configured to generate the control voltage (V_{ct1}) based on the comparison output (Vₑᵣᵣₒᵣ) from the voltage comparator 503 and on the bias voltage (V_{bias}) from the bias voltage generator 501. In this embodiment, the summing amplifier 500 includes an adder 502 coupled to the bias voltage generator 501 and the output end of the voltage comparator 503 for receiving the bias voltage (V_{bias}) and the comparison output (Vₑᵣᵣₒᵣ) therefrom, and an operational amplifier 504 coupled between the adder 502 and the voltage controlled current source 505 for generating the control voltage (V_{ct1}) based on the bias voltage (V_{bias}) and the comparison output (Vₑᵣᵣₒᵣ). In this case, the control voltage (V_{ct1}) can be expressed as the following equation:

V_{ct1}=m1×V_{bias}+(1−m1)×Vₑᵣᵣₒᵣ,

where m1 is a bias parameter.

In such a configuration of the IR emitter driver circuit 50, the voltage comparator 503 can maintain the voltage (V1) to be equal to the voltage (V2). That is to say, I×R_{ref}=kI×Rₑₘᵢₜₜₑᵣ, where R_{ref} represents the resistance of the reference sensing resistor 506 and Rₑₘᵢₜₜₑᵣ represents the resistance of the IR emitter 4, and hence Rₑₘᵢₜₜₑᵣ=R_{ref}/k. It is noted that, since the reference sensing resistor 506 has the fixed resistance (R_{ref}), the resistance (Rₑₘᵢₜₜₑᵣ) of the IR emitter 4 can be maintained at a fixed value of R_{ref}/k. On the other hand, the heater controller 52 can control the heater 53 to maintain the temperature in the receiving chamber 11 at the desired specific temperature. Therefore, the IR emitter 4 can normally operate at a fixed working temperature regardless of changes in the ambient temperature to thereby stabilize the IR spectrum radiation emitted thereby.

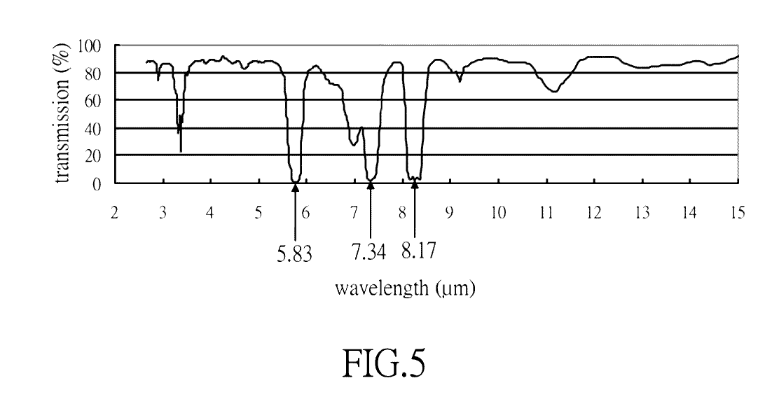

The IR detector 6 is disposed in the optical cavity 12 (FIG. 2), and is arranged so that the IR spectrum radiation emitted from the IR emitter 4 propagates to the IR detector 6 along the zigzag optical path between the IR emitter 4 and the IR detector 6. Due to the control unit 5, the IR detector 6 operates at the desired specific temperature to detect the IR spectrum radiation in the optical cavity 12 so as to generate an IR signal that has a wavelength within a wavelength range around a specific IR absorption wavelength of acetone. In this embodiment, the IR detector 6 includes a narrow bandpass filter 61 (FIG. 1) that has a pass band at the specific IR absorption wavelength of acetone with a bandwidth ranging from, for example, 90 nm to 180 nm. FIG. 5 illustrates the IR absorption spectrum for acetone. From FIG. 5, the specific IR absorption wavelength of acetone is, for example, 5.83 μm in this embodiment. However, in some embodiments, the specific IR absorption wavelength of acetone can be one of 7.34 μm and 8.17 μm.

The signal processing unit 7 is disposed in the housing 1, and is connected electrically to the IR detector 6 for receiving the IR signal therefrom. The signal processing unit 7 is configured to process and analyze the IR signal so as to obtain a measurement result indicative of the concentration of acetone gas contained in the gas sample. In this embodiment, the signal processing unit 7 includes an amplifying circuit 71, an analog-to-digital (A/D) converter 72 and a microprocessor 73 (FIG. 1).

The amplifying circuit 71 is connected electrically to the IR detector 6 for receiving and amplifying the IR signal therefrom.

The A/D converter 72 is connected to the amplifying circuit 71 for receiving the IR signal amplified by the amplifying circuit 71. The A/D converter 72 is configured to convert the amplified IR signal received thereby into a digital voltage output in a known high resolution manner. In this embodiment, the A/D converter 72 is, for example, a signal-delta-type A/D converter. Thus, the digital voltage output may have more than 14 bits.

The microprocessor 73 is connected electrically to the A/D converter 72 for receiving the digital voltage output therefrom. The microprocessor 73 is configured to analyze the digital voltage output to obtain the measurement result.

The display unit 8 is mounted to the housing 1, and is connected electrically to the microprocessor 73 (FIG. 1) for displaying the measurement result thereon.

In this embodiment, the microprocessor 73 further controls the outlet valve 15 during sampling of the gas sample so that the end tidal portion of the filtered exhaled breath is kept in the optical cavity 12 to serve as the gas sample. In addition, the microprocessor 73 further controls operation of the switch 508 of the IR emitter driver circuit 50. Referring again to FIG. 4, the microprocessor 73 is connected electrically to the control end of the switch 508 for outputting the control voltage (Vₒₙ) thereto.

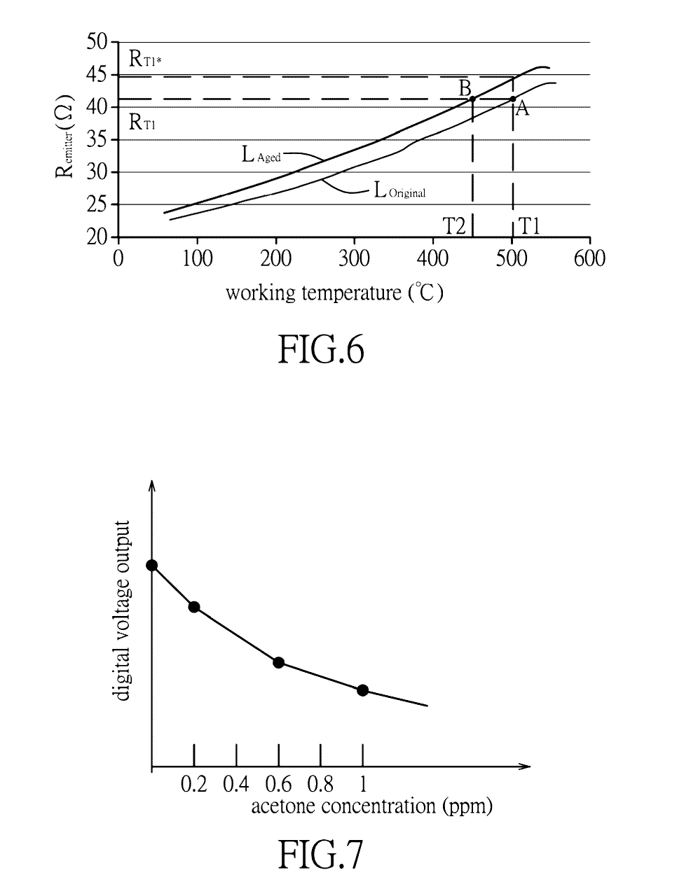

FIG. 6 illustrates an example of the relationships between the resistance (Rₑₘᵢₜₜₑᵣ) and the working temperature of the IR emitter 4 in an original state indicated by a thin curve (L_{Original}) and an aged state indicated by a thick curve (L_{Aged}). In this example, when in the original state, the IR emitter 4 may operate at the point (A), where the resistance (Rₑₘᵢₜₜₑᵣ) is R_{T1} and the working temperature is T1. When the IR emitter 4 has become aged after long-term operation, i.e., when in the aged state, the aged IR emitter 4 may have an increased resistance at room temperature of about 25° C. In this case, the resistance-to-working temperature characteristic curve of the aged IR emitter 4 may be shifted from the thin carve (L_{Original}) to the thick curve (L_{Aged}) such that the resistance changes from R_{T1} to R_{T1*} at the same working temperature of T1 (i.e., under the condition where the power applied to the emitter chip 41 of the IR emitter 4 remains unchanged). However, due to the constant-resistance control implemented for the IR emitter 4 by the IR emitter driver circuit 50, the aged IR emitter 4 may operate at the point (B), where the working temperature of the aged IR emitter 4 may be slightly lowered from T1 down to T2, such that the power applied to the aged IR emitter 4 cannot remain unchanged. Therefore, to maintain the same power, it is required to increase the driving current (kI) supplied by the mirrored current source 507 to the aged IR emitter 4 by increasing the bias voltage (V_{bias}) from the bias voltage generator 501 (FIG. 4).

In order to compensate for the aforesaid aging effect of the IR emitter 4, referring again to FIG. 4, the microprocessor 73 is further connected electrically to the common node (n1) and the bias voltage generator 501 of the IR emitter driver circuit 50. The microprocessor 73 is configured to monitor the reference current (I) supplied by the voltage controlled current source 505 and to output, upon detecting a variation in the reference current (I), a compensation control signal to the bias voltage generator 501. In this case, the bias voltage generator 501 adjusts the bias voltage in response to the compensation control signal from the microprocessor 73 to thereby compensate for the variation in the reference current (I) due to aging of the IR emitter 4. As a result, the microprocessor 73 can also act as an IR emitter aging compensator through fine adjustment of the driving current (kI) provided from the IR emitter driver circuit 50 to the IR emitter 4.

On the other hand, the control unit 5 further includes the optical radiation sensor 54 built in the IR emitter 4 (see FIGS. 3A and 3B) and coupled to the microprocessor 73 of the signal processing unit 7 (FIG. 1) through, for example, the required wiring and corresponding conductive leads 48 (FIG. 3A). As a result, refer ring again to FIG. 4, the microprocessor 73 receives the sensing output from the optical radiation sensor 54, and is configured to output, in response to the sensing output, a feedback control signal to the bias voltage generator 501 such that the bias voltage generator 501 adjusts the bias voltage (V_{bias}) in response to the feedback control signal to thereby maintain the power applied to the IR emitter 4 to be the same. In other words, the microprocessor 73 can control the bias voltage generator 501 based on the sensing output from the optical radiation sensor 54 to finely tune the bias voltage (V_{bias}) to thereby adjust the reference current (I) and the driving current (kI) for the aging compensation of the IE emitter 4. Therefore, the sensing output from the optical radiation sensor 54 can also be used as feedback control of the driving current (kI) for the IR emitter 4, thereby stabilizing the IR spectrum radiation emitted by the IR emitter 4.

If is noted that, in some embodiments, the optical radiation sensor 54 may be disposed in the optical cavity 12 for sensing the IR spectrum radiation emitted by the IR emitter 4 to generate the sensing output. Alternatively, the optical radiation sensor 54 may be incorporated together with the IR detector 6 into a single module.

For more accurate measurement of acetone concentration, the pressure sensor 9 is disposed in the optical cavity 12 (FIG. 2), and is connected electrically to the microprocessor 73 of the signal processing unit 7 (FIG. 1). The pressure sensor 3 is configured to sense the gas pressure in the optical cavity 12 to generate a pressure output indicative of the gas pressure in the optical cavity 12.

During the manufacturing phase, the portable NDIR breath acetone measurement apparatus is operated in a calibration mode. When in the calibration mode, several gas samples with known different acetone concentrations are successively fed to the optical cavity 12, and then the IR detector 6 generates IR signals corresponding respectively to the gas samples. Thus, the digital voltage outputs generated by the A/D converter 72 and corresponding respectively to the gas samples with the different acetone concentrations can be obtained as indicated by the black points in FIG. 7. The pressure value obtained during the calibration mode is used for pressure compensation in real measurement condition according to ideal gas law. Thereafter, the signal processing unit 7 conducts, based on the IR signals from the IR detector 6, an operation of a polynomial fit curve to determine some estimation parameters of the polynomial fit curve. FIG. 7 illustrates an example of the polynomial fit curve obtained by the embodiment when operating in the calibration mode. In FIG. 7, acetone concentrations corresponding respectively to the black points represent the known acetone concentrations of the gas samples.

During measurement, the microprocessor 73 receives the digital voltage output from the A/D converter 72 that is associated with the IE signal from the IR detector 6, and uses the polynomial fit curve shown in FIG. 7 to get a pre-pressure corrected gas concentration value. The microprocessor 73 further receives the pressure output from the pressure sensor 9, and adjusts the measurement result obtained thereby based on the current pressure output and a predetermined calibrated gas pressure according to ideal gas law.

To sum up, since the control unit 5 can provide quick response to maintain the temperature in the receiving chamber 11 at the desired specific temperature, the IR emitter 4 can operate at a fixed working temperature during measurement. In particular, the IR emitter driver circuit 50 implements constant-resistance driving for the IR emitter 4 through quick analog feedback control. In addition, the bias voltage generator 501 cooperates with the optical radiation sensor 54 and/or the microprocessor 73 to achieve the aging compensation of the IR emitter 4. Furthermore, due to the presence of the pressure sensor 9 for compensating for pressure change in the optical cavity 12, accurate acetone measurement can be achieved. Therefore, the portable NDIR breath acetone measurement apparatus of this disclosure can realize acetone measurement with sub-ppm accuracy. Moreover, since the portable NDIR breath acetone measurement apparatus of this disclosure uses inexpensive components and can be fabricated to have a compact size, it is suitable for point-of-care or home use in non-invasive diabetes monitoring or fat loss monitoring of healthy human beings.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A portable non-dispersive infrared (NDIR) breath acetone measurement apparatus comprising:
a portable housing configured with a receiving chamber that is formed with an optical cavity for containing a gas sample taken from an exhaled breath of a human subject;
an IR emitter disposed in said receiving chamber for emitting IR spectrum radiation at an IR absorption band of acetone into said optical cavity;
a control unit connected electrically to said IR emitter, said control unit being configured to control the temperature in said receiving chamber to a desired specific temperature that is higher than ambient temperature of said housing and to control said IR emitter to stabilize the IR spectrum radiation emitted thereby;
an IR detector disposed in said optical cavity, and arranged so that the IR spectrum radiation emitted from said IR emitter propagates to said IR detector along a zigzag optical path defined between said IR emitter and said IR detector, said IR detector being operated at the desired specific temperature to detect the IR spectrum radiation in said optical cavity so as to generate an IR signal that has a wavelength within a wavelength range around a specific IR absorption wavelength of acetone; and
a signal processing unit connected electrically to said IR detector tor receiving the IR signal therefrom, said signal processing unit being configured to process and analyze the IR signal so as to obtain a measurement result indicative of the concentration of acetone gas contained in the gas sample.

2. The NDIR portable breath acetone measurement apparatus as claimed in claim 1, further comprising:
a mouth piece mounted to said housing for collecting the exhaled breath of the human subject; and
a water vapor absorption filter connected fluidly to said mouth piece and said optical cavity for filtering water vapor out of the exhaled breath collected through said mouth piece;
wherein said receiving chamber has an inlet in fluid communication between said water vapor absorption filter and said optical cavity for the inflow of the exhaled breath filtered by said water vapor absorption filter and entering said optical cavity, and an outlet in fluid communication with said optical cavity for the outflow of the gas sample from said optical cavity.

3. The portable NDIR breath acetone measurement apparatus as claimed in claim 2, wherein:
said outlet of said receiving chamber is provided with an outlet valve; and
said signal processing unit further controls said outlet valve in a manner that, during sampling of the gas sample from the exhaled breath filtered by said water vapor absorption filter, an end tidal portion of the exhaled breath filtered by said water vapor absorption filter is kept in said optical cavity to serve as the gas sample.

4. The portable NDIR breath acetone measurement apparatus as claimed in claim 1, wherein said optical cavity is defined by a highly IR-reflective inner wall surface of said housing.

5. The portable NDIR breath acetone measurement apparatus as claimed in claim 4, wherein said highly IR-reflective inner wail surface is coated with gold (Au).

6. The portable NDIR breath acetone measurement apparatus as claimed in claim 1, wherein the length of the zigzag optical path is greater than 200 cm.

7. The portable NDIR breath acetone measurement apparatus as claimed in claim 1, wherein said IR emitter is a microelectromechanical systems (MEMS)-based IR emitter.

8. The portable NDIR breath acetone measurement apparatus as claimed in claim 7, wherein said IR emitter includes:
an emitter chip for emitting IR radiation;
an enclosure for housing said emitter chip therein, said enclosure having a metal base, and a metal cap connected to said metal base and formed with a window opening; and
an IR filter attached internally to said metal cap to cover sealingly said window opening, said IR filter being configured to filter the IR radiation emitted by said emitter chip so as to allow the IR spectrum radiation at the absorption band of acetone to radiate out of said enclosure through said window opening.

9. The portable NDIR breath acetone measurement apparatus as claimed in claim 1, where in said IR detector includes a narrow bandpass filter that has a pass band at the specific IR absorption wavelength of acetone with a bandwidth ranging from 90 nm to 180 nm.

10. The portable NDIR breath acetone measurement apparatus as claimed in claim 9, wherein the specific IR absorption wavelength of acetone is one of 5.83 μm, 7.34 μm and 8.17 μm.

11. The portable NDIR breath acetone measurement apparatus as claimed in claim 1, wherein said signal processing unit includes:
an amplifying circuit connected electrically to said IE detector for receiving and amplifying the IR signal therefrom;
an analog-to-digital (A/D) converter connected electrically to said amplifying circuit for receiving the IR signal amplified by said amplifying circuit, said A/D converter being configured to convert the IR signal received thereby into a digital voltage output in a high resolution manner; and
a microprocessor connected electrically to said A/D converter for receiving the digital voltage output therefrom, said microprocessor being configured to analyze the digital voltage output to obtain the measurement result.

12. The portable NDIR breath acetone measurement apparatus as claimed in claim 11, wherein said A/D converter is a sigma-delta-type A/D converter, and the digital voltage output has more than 14 bits.

13. The portable NDIR breath acetone measurement apparatus as claimed in claim 11, further comprising a display unit mounted to said housing, and connected electrically to said microprocessor for displaying the measurement result thereon.

14. The portable NDIR breath acetone measurement apparatus as claimed in claim 11, further comprising
a pressure sensor disposed in said optical cavity and connected electrically to said microprocessor of said signal processing unit, said pressure sensor being configured to sense the gas pressure in said optical cavity to generate a pressure output indicative of the gas pressure in said optical cavity,
wherein said microprocessor of said signal processing unit further receives the pressure output from said pressure sensor, and adjusts the measurement result obtained thereby based on the pressure output and a predetermined calibrated gas pressure upon detecting that the gas pressure in said optical cavity differs from the predetermined calibrated gas pressure to thereby compensate for pressure change in said optical cavity.

15. The portable NDIR breath acetone measurement apparatus as claimed in claim 11, wherein said portable NDIR breath acetone measurement, apparatus is capable of operating in a calibration mode, where said IR detector generates a plurality of calibrated IR signals that correspond respectively to different concentrations of calibrated acetone gas contained in said optical cavity, and where said microprocessor of said signal processing unit is configured to generate, based on the calibrated IR signals and the different concentrations of the calibrated acetone gas, an estimation equation to be used to estimate the concentration of acetone gas contained in the gas sample.

16. The portable NDIR breath acetone measurement apparatus as claimed in claim 1, wherein said control unit includes:
a temperature sensor disposed in said receiving chamber for sensing the temperature in said receiving chamber to generate an output indicative of the temperature in said receiving chamber;
a heater disposed in said receiving chamber for heating said receiving chamber; and
a heater controller connected electrically to said temperature sensor and said heater, said heater controller receiving the output from said temperature sensor, and controlling said heater based on the output to maintain the temperature in said receiving chamber at the desired specific temperature.

17. The portable NDIR breath acetone measurement apparatus as claimed in claim 1, wherein said control unit includes an IR emitter driver circuit coupled to said IR emitter for providing a driving current thereto, said IR emitter driver circuit including:
a voltage controlled current source for generating a reference current based on a control voltage;
a mirrored current source coupled to said IR emitter for supplying thereto the driving current that is k times the reference current;
a reference sensing resistor coupled between said voltage controlled current source and ground to allow the reference current from said voltage controlled current source to flow therethrough;
a voltage comparator for comparing a voltage across said reference sensing voltage and a voltage across said IR emitter to generate a comparison output;
a bias voltage generator for general lug a bias voltage; and
a summing amplifier coupled to said voltage controlled current source, said voltage comparator and said bias voltage generator, said summing amplifier being configured to generate the control voltage based on the comparison output from said voltage comparator and the bias voltage from said bias voltage generator.

18. The portable NDIR breath acetone measurement apparatus as claimed in claim 17, wherein said signal processing unit includes a microprocessor that is coupled to said voltage controlled current source, said IR emitter and said bias voltage generator, said microprocessor being configured to monitor the reference current supplied by said voltage controlled current source and to output; upon detecting a variation in the reference current, a compensation control signal to said bias voltage generator, such that said bias voltage generator adjusts the bias voltage in response to the compensation control signal to thereby compensate for the variation in the reference current due to aging of said IR emitter.

19. The portable NDIR breath acetone measurement apparatus as claimed in claim 18, wherein said control unit further includes a switch coupled between said IR emitter and ground, said switch being controlled by said microprocessor of said signal processing unit to allow supply of the driving current from said mirrored current source to said IR emitter.

20. The portable NDIR breath acetone measurement apparatus as claimed in claim 17, wherein:
said control unit further includes an optical radiation sensor disposed in said optical cavity for sensing the IR spectrum radiation emitted by said IS emitter to generate a sensing output; and
said signal processing unit includes a microprocessor coupled to said optical radiation sensor and said bias voltage generator, said microprocessor receiving the sensing output from said optical radiation sensor and being configured to output, in response to the sensing output, a feedback control signal to said bias voltage generator such that said bias voltage generator adjusts the bias voltage in response to the feedback control signal to thereby maintain the power applied on said IR emitter to be the same.
